(19) United States
(12) Patent Application Publication
(10) Pub. No.: US 2022/0073972 A1
(43) Pub. Date: Mar. 10, 2022

(54) METHOD FOR SIMULTANEOUSLY DETECTING EXOSOME MEMBRANE PROTEIN AND MRNA

(71) Applicant: HANGZHOU DIXIANG CO.,LTD, Zhejiang (CN)

(72) Inventor: Hengshan ZENG, Zhejiang (CN)

(73) Assignee: HANGZHOU DIXIANG CO.,LTD, Zhejiang (CN)

(21) Appl. No.: 17/415,747

(22) PCT Filed: May 31, 2019
(86) PCT No.: PCT/CN2019/089478

§ 371 Date: Jun. 18, 2021

(30) Foreign Application Priority Data
Dec. 29, 2018 (CN) 201811636332.3

(51) Int. Cl. C12Q 1/6816; C12Q 1/6834

(52) U.S. Cl. CPC C12Q 1/6816; C12Q 1/6834

(57) ABSTRACT

A method for simultaneously detecting an exosome membrane protein and mRNA is provided. The method can perform simultaneous detection on exosomes separated and purified from the same sample by labeling an exosome membrane protein with a fluorescent antibody and labeling a target gene mRNA with a molecular beacon, wherein labeling the exosome with the molecular beacon is specifically performed by means of an in-situ exosome capture well plate or chip, and each well or chip in the in-situ exosome capture well plate includes a fluorescein-labeled molecular beacon; and the molecular beacon is a specific DNA probe for detecting the target gene mRNA. The present invention utilizes an in-situ exosome capture well plate or chip technology to detect a biomarker gene mRNA contained in an exosome of a biological sample.

### BACKGROUND

### Technical Field

The present invention belongs to the technical field of detection, and specifically relates to a method for simultaneously detecting an exosome membrane protein and mRNA.

### Description of Related Art

Exosome is a kind of microvesicle capable of being secreted by most of cells, and has a diameter of about 30-150 nm, and a phospholipid bilayer structure, and can protect substances coated thereby well. Such kind of microvesicle contains specific proteins, nucleic acid and lipid of a host cell source, and can be used as a signaling molecule to be delivered to other cells; and microvesicle is an important communication medium among cells, and thus can render receptor cells to make multiple biological function changes. All cells can produce exosomes, and the exosomes secreted by different cells have different components and content; a specific gene product is selectively loaded into an exosome, thus participating in biological function regulation of a receptor cell by transferring bioactive molecules among different cells. The exosome is most featured by rich content, specific and stable inclusion sources and thus, exosome has gradually become a hot topic in the research field of cytobiology.

In-situ exosome capture well plate and chip technology is a brand-new in-situ exosome capture and detection technology, specifically in the detection of mRNA and microRNA in exosome, and exosome membrane proteins. With an activated biomolecule membrane-coated glass as a carrier, a cationic lipid nanoparticle coated with a molecular beacon (self-designed) specifically recognizing mRNA or microRNA of a target gene is fused with a negatively-charged exosome; and then the molecular beacon is bonded with the target, and the specific fluorescent antibody is bonded with a membrane protein on a fusion body to produce a fluorescence signal under the stimulation of a laser; then the obtained product is detected by total internal reflection fluorescence (TIRF); signal strength is in direct proportion to the content of the corresponding target, thus judging the course of a disease or locking pathogen. TIRF imaging has ultramicro feature and is ultrasensitive to the fluorescence signal; therefore, the exosome capture well plate or a chip is combined with the TIRF imaging technology to achieve the direct imaging to exosome, such a nanoscale vesicle and the semi-quantitative detection of the inclusion thereof. Since there are a large number of exosomes in various biological samples, and they are rich in specific nucleic acid from a specific cell, the exosome therein is separated, and identified by a high-sensitivity exosome capture well plate and a chip detection technology. The detection for an exosome membrane protein PDL1 of a tumor cell source and mRNA thereof is set as an example; and the detection principle is shown in FIG. 1.

The technology has the following features:

1. in a biological sample, exosome is negatively charged, and its capsule has a structure similar to cytomembrane;

2. the lipid nanoparticle containing a specific molecular beacon made by ourselves is positively charged itself; and its capsule is fully close to the structure of cytomembrane;

3. attracted by positive and negative charges, exosome contacts with nanoparticles on a well plate or chip very easily, and both are subjected to membrane fusion to form a lipid membrane complex, and to rapidly achieve charge and volumetric balance; and at this time, no more exosome is fused any more, thus achieving the in-situ and quantitative capture of exosome;

4. after fusion, exosome is mixed with an inclusion of the nanoparticle; a specific molecular beacon renders the exosome to hybridize with the target gene mRNA or microRNA to emit green fluorescence under the stimulation of a laser;

5. meanwhile, the original membrane protein of exosome will be redistributed, but its antigenicity will be not influenced; when the antigen and a specific fluorescent antibody are subjected to a warm bath to achieve specific binding, thus emitting orange-yellow fluorescence under the stimulation of a laser;

6. in such way, an experimental process achieves the synchronous detection of a membrane protein and target gene microRNA of an exosome from a sample source.

Based on the experiment demand, the exosome capture well plate or chip can be made into multiple specifications of 24, 48, 96, and 384-well; and each well can be coated with a single or multiple fluorescein-labeled molecular beacons; then multiple target gene detection channels are integrated onto a piece of well plate or a chip, thereby beneficial to multi-sample and high-throughput screening; therefore, the present invention is rapid, convenient, economic, and has significant advantages.

### SUMMARY

Directed to the problem existing in the prior art, the objective of the present invention is to design and provide a technical solution of a method for simultaneously detecting exosome membrane protein and mRNA.

The method for simultaneously detecting exosome membrane protein and mRNA is characterized in that the method can perform simultaneous detection on exosomes separated and purified from the same sample by labeling an exosome membrane protein with a fluorescent antibody and labeling a target gene mRNA with molecular beacon; where labeling the exosome with the molecular beacon is specifically performed by means of an in-situ exosome capture well plate or chip; and each well or chip in the in-situ exosome capture well plate includes a fluorescein-labeled molecular beacon; and the molecular beacon is a specific DNA probe for detecting the target gene mRNA.

The method for simultaneously detecting exosome membrane protein and mRNA is characterized in that a 5′ stem and loop on the specific DNA probe are completely complementary to the target gene; and a 3′ stem is partially complementary to the 5′ stem; a 5′ terminal and a 3′ terminal are respectively modified by a fluorophore and a quencher group; and partial bases on the loop are modified by locked nucleic acid.

The method for simultaneously detecting exosome membrane protein and mRNA is characterized in that the specific DNA probe is self-designed and modified for synthesis according to a target gene sequence; and the fluorescent antibody for labeling the exosome membrane protein is a fluorescein-labeled monoclonal antibody.

The method for simultaneously detecting exosome membrane protein and mRNA is characterized in that the specific DNA probe is coated with a cationic lipid composite nanoparticle.

Application of a fluorescent antibody of the specific exosome membrane protein and a specific DNA probe of exosome target gene mRNA is in the detection of exosome of a specific sample source and in a scientific experiment with a clear target.

The application is characterized in that the specific sample includes: cell culture supernatants, isolated laboratory animal plasma, serum, isolated human plasma, serum, urine, and other body fluid or excrement samples.

The application is characterized in that the scientific experiment aims at detecting a membrane protein and mRNA of exosome from a source of a living cell, an animal, a human body fluid or an excrement sample.

The application is characterized in that the fluorescent antibody for detecting a specific membrane protein of exosome is a fluorescein-labeled monoclonal antibody.

The application is characterized in that a 5′ stem and loop on the specific DNA probe are completely complementary to a target gene; and a 3′ stem is partially complementary to the 5′ stem; a 5′ terminal and a 3′ terminal are respectively modified by a fluorophore and a quencher group; and partial bases on the loop are modified by locked nucleic acid.

The application is characterized in that the specific DNA probe is self-designed according to a target gene sequence and modified for synthesis. The present invention utilizes an in-situ exosome capture well plate and chip technology, and detects the exosome membrane protein and mRNA from a specific sample source simultaneously, and can be used in various scientific experiments associated with exosomes. The technology is a novel detection technology based on exosome membrane protein and mRNA, and has the advantages of ultra-sensitivity, rapidness, specificity, and the like.

### BRIEF DESCRIPTION OF THE DRAWINGS

FIG. 1 is a schematic diagram showing the detection principle of an exosome membrane protein PDL1 and mRNA thereof as an example;

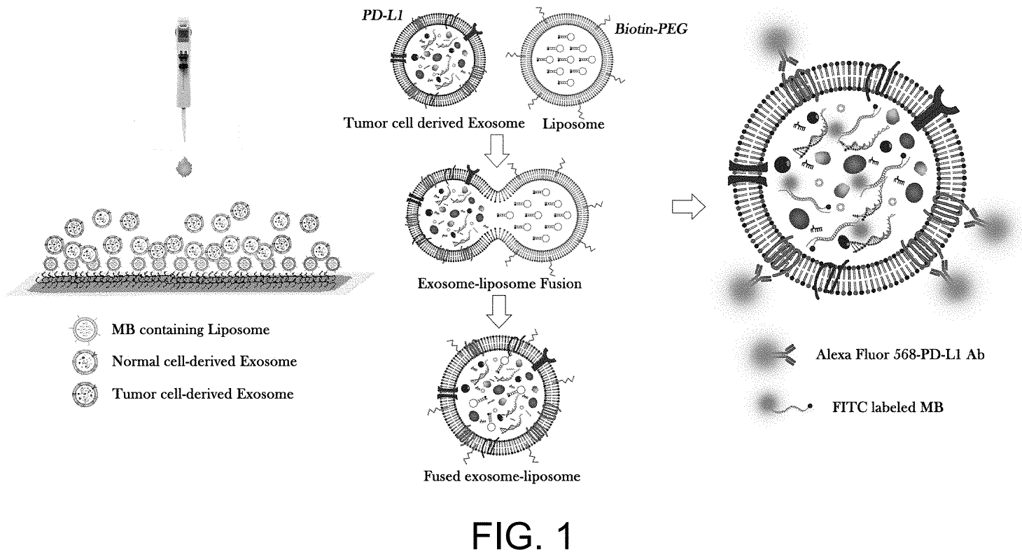

FIG. 2 shows expression of a membrane protein PDL1 and mRNA of Example 2 in an exosome of a lung cell line;

FIG. 3 shows expression of the membrane protein PDL1 and mRNA of Example 2 in an exosome of a lung tumor;

FIG. 4 shows expression of the membrane protein GP73 and mRNA of Example 2 in an exosome of a liver cell line;

FIG. 5 shows expression of a membrane protein GP73 and mRNA of Example 2 in an exosome of a liver tumor;

### DESCRIPTION OF THE EMBODIMENTS

The present invention is further described in combination with embodiments below.

### Example 1: Design of a Specific Molecular Beacon (Targets PDL1 and GP73 were Set as Examples)

It is crucial to design a specific molecular beacon for detecting a target gene for an exosome capture well plate or chip to detect specific nucleic acid. For this purpose, the Applicant designed a molecular beacon having a special stem-loop structure in combination with the features of a target gene; a 5′ stem and loop were completely complementary to the target gene; a 3′ stem was partially complementary to the 5′ stem; a 5′ terminal and 3′ terminal were respectively modified by a fluorophore and a quencher group; and partial bases on the loop were modified by locked nucleic acid; the specific PDL1 molecular beacon had a specific sequence as shown in Table 1; the sequence shown in SEQ ID No. 1 had the base modification mode: base 1 was modified by 6FAM; bases 10, 13, 16, 19, 22, 25 and 28 were modified by LNA, and base 36 was modified by BHQ1; the sequence shown in SEQ ID No. 2 had the base modification mode: base 1 was modified by 6FAM; bases 10, 13, 16, 19, 22, 25 and 28 were modified by LNA, and base 35 was modified by BHQ1; the sequence shown in SEQ ID No. 3 had the base modification mode: base 1 was modified by 6FAM; bases 10, 13, 16, 19, 22, and 25 were modified by LNA, and base 34 was modified by BHQ1; the sequence shown in SEQ ID No. 4 had the base modification mode: base 1 was modified by 6FAM; bases 10, 13, 16, 19, 22, 25 and 28 were modified by LNA, and base 35 was modified by BHQ1; the sequence shown in SEQ ID No. 5 had the base modification mode: base 1 was modified by 6FAM; bases 10, 13, 16, 19, 22, 25 and 28 were modified by LNA, and base 35 was modified by BHQ1; and the sequence shown in SEQ ID No. 6 had the base modification mode: base 1 was modified by 6FAM; bases 10, 13, 16, 19, 22, 25 and 28 were modified by LNA, and base 36 was modified by BHQ1. For this purpose, the Applicant designed a molecular beacon having a special stem-loop structure in combination with the features of a target gene; a 5′ stem and loop were completely complementary to the target gene; a 3′ stem was partially complementary to the 5′ stem; a 5′ terminal and 3′ terminal were respectively modified by a fluorophore and a quencher group; and partial bases on the loop were modified by locked nucleic acid; the specific PDL1 molecular beacon had a specific sequence as shown in Table 1; the sequence shown in SEQ ID No. 1 had the base modification mode: base 1 was modified by 6FAM; bases 10, 13, 16, 19, 22, 25 and 28 were modified by LNA, and base 36 was modified by BHQ1; the sequence shown in SEQ ID No. 3 had the base modification mode: base 1 was modified by 6FAM; bases 10, 13, 16, 19, 22, 25 and 28 were modified by LNA, and base 35 was modified by BHQ1; the sequence shown in SEQ ID No. 3 had the base modification mode: base 1 was modified by 6FAM; bases 10, 13, 16, 19, 22, and 25 were modified by LNA, and base 40 was modified by BHQ1; the sequence shown in SEQ ID No. 5 had the base modification mode: base 1 was modified by 6FAM; bases 10, 13, 16, 19, 22, 25 and 28 were modified by LNA, and base 35 was modified by BHQ1; the sequence shown in SEQ ID No. 5 has the base modification mode: base 1 was modified by 6FAM; bases 10, 13, 16, 19, 22, 25 and 28 are modified by LNA, and base 35 was modified by BHQ1; the sequence shown in SEQ ID No. 6 has the base modification mode: base 1 was modified by 6FAM; bases 10, 13, 16, 19, 22, 25 and 28 were modified by LNA, and base 36 was modified by BHQ1; and the sequence shown in SEQ ID No. 7 has the base modification mode: base 1 was modified by 6FAM; bases 10, 13, 16, 19, 22, 25 and 28 were modified by LNA, and base 36 was modified by BHQ1.

The specific molecular beacon designed by the present invention improves the binding specificity of a molecular beacon to a target gene to the maximum extent, and reduces the background fluorescence intensity of the reaction. After the molecular beacon was synthesized, to prove its binding specificity to the corresponding target gene and optimum working temperature, we designed the following Table 3, thus choosing the optimum molecular beacon based on the maximum Signal to Noise Ratio (SNR), and its working temperature.

TABLE 1
PDL1 probe sequence

| | Sequence (5′-3′, “+” represented that LNA | Modifying |
|---|---|---|
| No. | modified base) | group |
| 1 | 5′ | 6FAM/ |
| | 6FAM-CGCGATCGG+AGG+ATG+TGC+CAG+AGG+TAG+TTGATCGCG- | BHQ1/ |
| | BHQ1 3′ (as shown in SEQ ID No. 1) | LNA |
| 2 | 5′ | 6FAM/ |
| | 6FAM-CGCGATCGC+TAT+GGT+GGT+GCC+GAC+TAC+AGATCGCG- | BHQ1/ |
| | BHQ1 3′ (as shown in SEQ ID No. 2) | LNA |
| 3 | 5′ | 6FAM/ |
| | 6FAM-CGCGATCTG+GTG+CCG+ACT+ACA+AGC+GAAGATCGCG- | BHQ1/ |
| | BHQ1 3′ (as shown in SEQ ID No. 3) | LNA |
| 4 | 5′ | 6FAM/ |
| | 6FAM-CGCGATCTG+GTG+CCG+ACT+ACA+AGC+GAA+TGATCGCG- | BHQ1/ |
| | BHQ1 3′ (as shown in SEQ ID No. 4) | LNA |
| 5 | 5′ | 6FAM/ |
| | 6FAM-CGCGATCGG+AGG+ATG+TGC+CAG+AGG+TAG+TGATCGCG- | BHQ1/ |
| | BHQ1 3′ (as shown in SEQ ID No. 5) | LNA |
| 6 | 5′ | 6FAM/ |
| | 6FAM-CGCGATCGC+TAT+GGT+GGT+GCC+GAC+TAC+AAGATCGCG- | BHQ1/ |
| | BHQ1 3′ (as shown in SEQ ID No. 6) | LNA |

TABLE 2
GP73 probe sequence

| | Sequence (5′-3′, “+” represented that LNA modified | Modifying |
|---|---|---|
| No. | base) | group |
| 1 | 5′ | 6FAM/ |
| | 6FAM-CGCGATCGG+CGG+CGA+CTT+CAT+GCT+GCG+AGATCGCG- | BHQ1/ |
| | BHQ1 3′ (as shown in SEQ ID No. 7) | LNA |
| 2 | 5′ | 6FAM/ |
| | 6FAM-CGCGATCGA+CTT+CAT+GCT+GCG+ACG+CCC+GTT+TGATCGCG- | BHQ1/ |
| | BHQ1 3′ (as shown in SEQ ID No. 8) | LNA |
| 3 | 5′ | 6FAM/ |
| | 6FAM-CGCGATCCG+CCC+TGC+GGA+CCC+TGC+CTT+CGATCGCG- | BHQ1/ |
| | BHQ1 3′ (SEQ ID No. 9) | LNA |
| 4 | 5′ | 6FAM/ |
| | 6FAM-CGCGATCCC+AGG+GCT+GCT+TGC+TTG+TCT+GTC+TCAGATCGCG- | BHQ1/ |
| | BHQ1 3′ (as shown in SEQ ID No. 10) | LNA |
| 5 | 5′ | 6FAM/ |
| | 6FAM-CGCGATCTG+CCA+GGG+CTG+CTT+GCT+TGT+CTG+TGATCGCG- | BHQ1/ |
| | BHQ1 3′ (as shown in SEQ ID No. 11) | LNA |
| 6 | 5′ | 6FAM/ |
| | 6FAM-CGCGATCGC+GAC+GCC+CGT+TTC+CCA+AGC+CGATCGCG- | BHQ1/ |
| | BHQ1 3′ (as shown in SEQ ID No. 12) | LNA |
| 7 | 5′ | 6FAM/ |
| | 6FAM-CGCGATCGC+TGC+GAC+GCC+CGT+TTC+CCA+AGGATCGCG- | BHQ1/ |
| | BHQ1 3′ (as shown in SEQ ID No. 13) | LNA |

TABLE 3

| | 37° C. | | | 42° C. | | | 50° C. | | | 55° C. | | |
|---|---|---|---|---|---|---|---|---|---|---|---|---|
| Pure | Tem- | Mole- | Template + | Tem- | Molecular | Template + | Tem- | Molecular | Template + | Tem- | Molecular | Template + |
| water* | plate | cular | Molecular | plate | beacon | Molecular | plate | beacon | Molecular | plate | beacon | Molecular |
| | | beacon | beacon | | | beacon | | | beacon | | | beacon |
| Body | An | An | An | An | An | An | An | An | An | An | An | An |
| fluid** | exosome | exosome | exosome | exosome | exosome | exosome | exosome | exosome | exosome | exosome | exosome | exosome |
| | capture | capture | capture | capture | capture | capture | capture | capture | capture | capture | capture | capture |
| | well | well | well | well | well | well | well | well | well | well | well | well |
| | plate | plate | plate | plate | plate | plate | plate | plate | plate | plate | plate | plate |
| | or chip | or chip | or chip | or chip | or chip | or chip | or chip | or chip | or chip | or chip | or chip | or chip |
| | + a | + a | + a | + a | + a | + a | + a | + a | + a | + a | + a | + a |
| | control | test | test | control | test | test | control | test | test | control | test | test |
| | sample | sample | sample | sample | sample | sample | sample | sample | sample | | sample | sample 2 |
| | | 1 | 2 | | 1 | 2 | | 1 | 2 | | 1 | |

*A fluorescent reader was used to read the fluorescence intensity.
**A TIRF microscope was used to detect the fluorescence intensity.

### Example 2: Detection Test (Targets PDL1 and GP73 were Respectively Set as Examples)

I. Exosome Isolation

1. 200 ul sample was taken (a cell culture supernatant, an isolated laboratory animal plasma, serum, isolated human plasma, serum, urine, and other body fluid or excrement sample), and 12000×g were centrifuged for 30 min at room temperature to remove cells and fragments;

2. supernatant was transferred to a new EP tube, and a 100 ul exosome precipitate reagent was added;

3. the above materials were mixed evenly for incubation for 30 min at 4° C.;

4. 10,000×g were centrifuged for 30 min at room temperature;

5. supernatant was removed by suction, and then 100 ul 1×PBS was taken to resuspend the precipitate rich in the exosome, standing at 4° C. for further use.

II. Purification of an Exosome Chromatographic Column

1. Chromatographic column balancing: 100 ul equilibrium liquid was added and 9000×g were centrifuged for 1 min;

2. sample loading: 100 ul resuspending solution was put on a column; and 9000×g were centrifuged for 1 min;

3. eluting: a 50 ul eluent was added, and 9000×g were centrifuged for 1 min.

III. Detection of an Exosome Capture Well Plate

1. The well plate or chip (each well in the well plate or chip can be coated with multiple fluorescein-labeled molecular beacons shown in Table 1 or Table 2, and the molecular beacon was coated with composite cationic lipid nanoparticles); and then the purified exosome eluent was added to sample wells;

2. negative and positive controls (negative and positive controls were a nematode gene segment and a target gene fragment respectively coated with nanoparticles) were added to the subsequent sample wells;

3. a PDL1 or GP73 fluorescent antibody was added according to a volume ratio of 1:1000;

4. the above materials were incubated for 1 h at 42° C.;

5. The well plate was washed by 1×PBS for three times, and an TIRF microscope was used to collect fluorescence images;

6. DXimageV1 software was used to analyze the images to configure a cut-off value automatically, and interpret the result of the sample to be tested automatically.

IV. Test Results

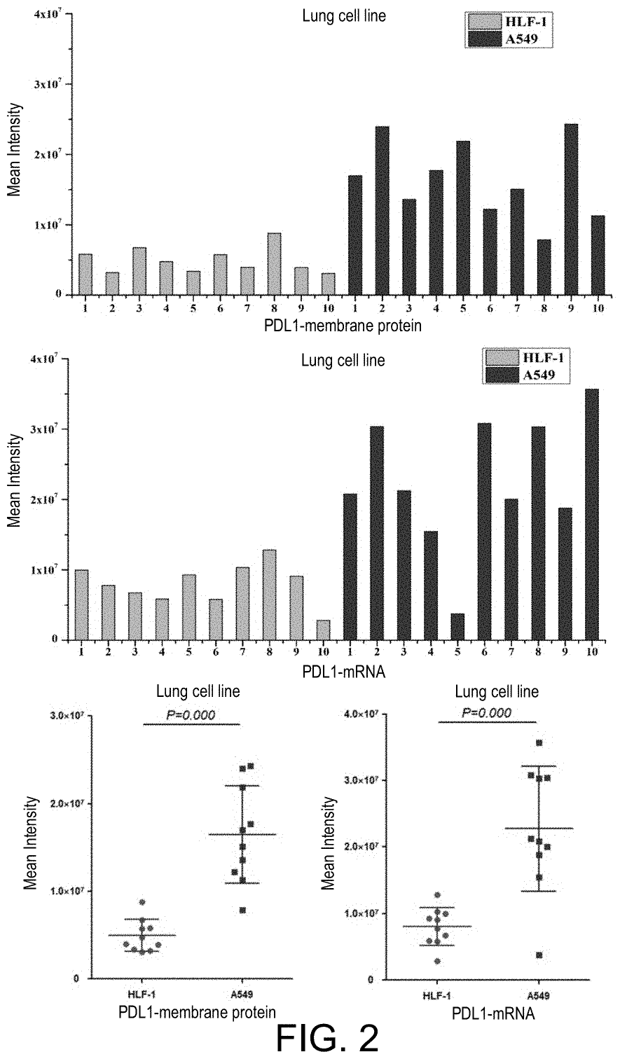

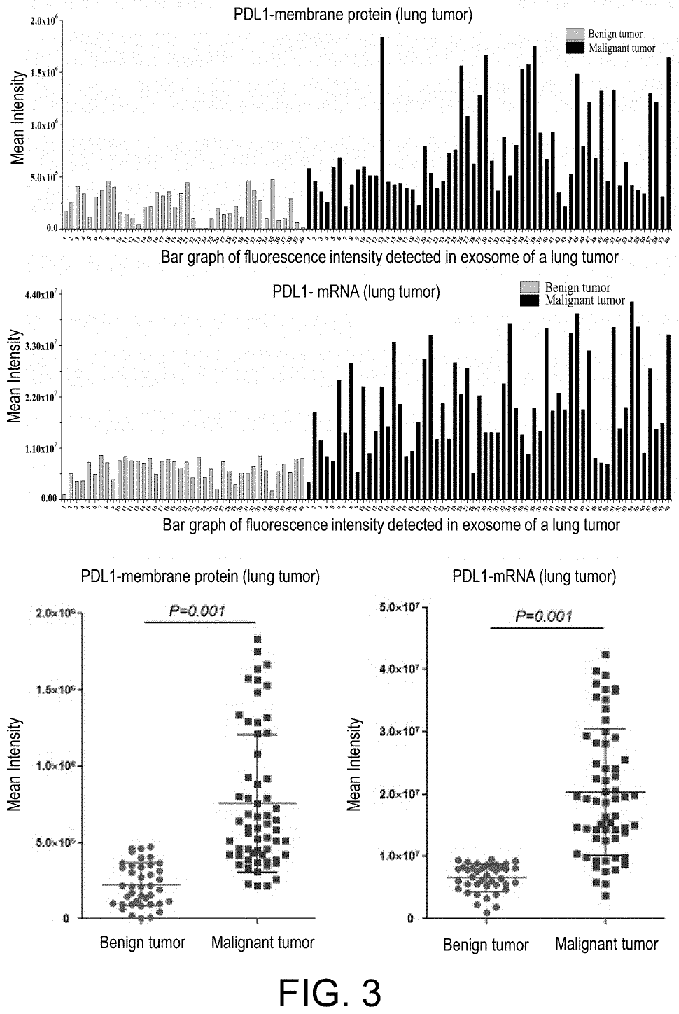

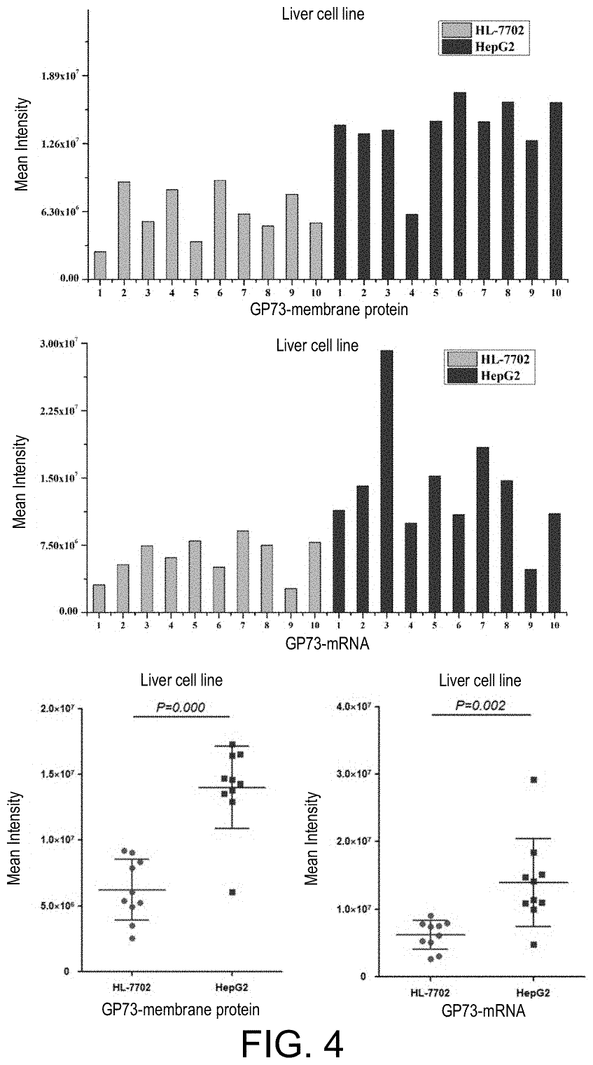

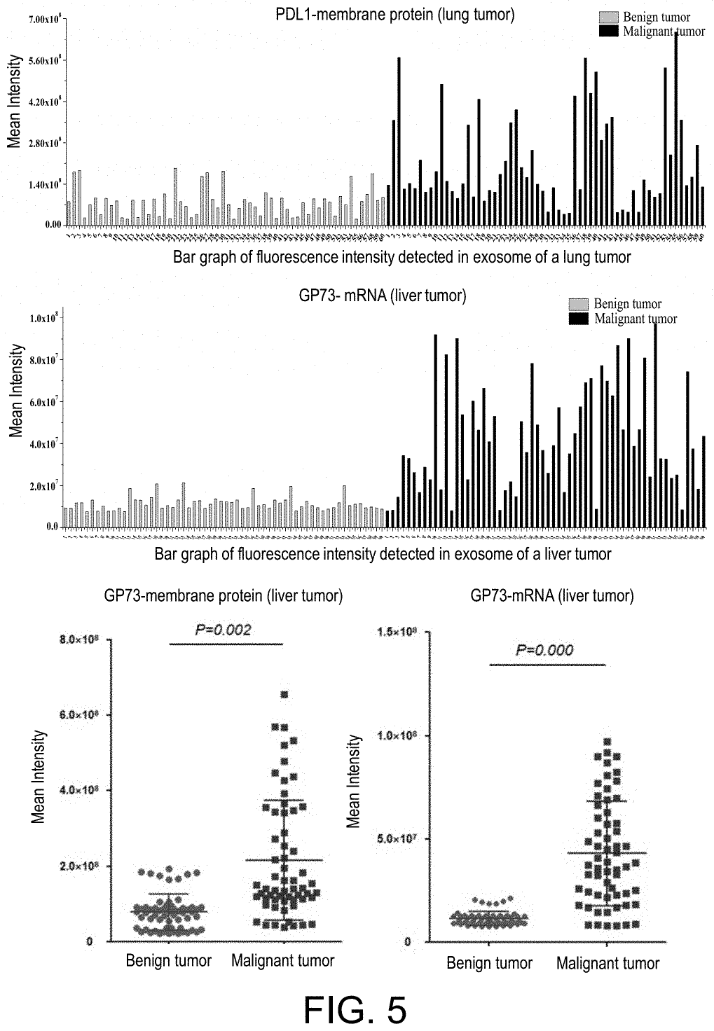

60 cases from a conventional normal human hepatic cell line HL-7702, a hepatoma carcinoma cell line HepG2, a normal lung cell line HLF-1 and a lung carcinoma cell line A549, and plasma samples of a patient suffering benign and malignant liver and lung tumors were configured respectively; and the results were shown in FIGS. 2, 3, 4, and 5; after the exosome capture well plate or chip was imaged under a TIRF microscope, the total fluorescence intensity of the exosome PDL1 membrane protein and mRNA from the lung carcinoma cell line A549 and malignant lung tumor was higher than that of the normal lung cell line HLF-1 and benign lung tumor. The total fluorescence intensity of the exosome GP73 membrane protein and mRNA from the hepatoma carcinoma cell line HepG2 and malignant liver tumor was higher than that of the normal lung cell line HL-7702 and benign liver tumor, indicating that the technology can simultaneously detect the target membrane protein and mRNA of the exosome.

1. A method for simultaneously detecting exosome membrane protein and mRNA, wherein the method can perform simultaneous detection on exosomes separated and purified from the same sample by labeling an exosome membrane protein with a fluorescent antibody and labeling a target gene mRNA with a molecular beacon; wherein labeling the exosome with the molecular beacon is specifically performed by means of an in-situ exosome capture well plate or chip, and each well or chip in the in-situ exosome capture well plate comprises a fluorescein-labeled molecular beacon; and the molecular beacon is a specific DNA probe for detecting the target gene mRNA.

2. The method for simultaneously detecting exosome membrane protein and mRNA of claim 1, wherein a 5′ stem and loop on the specific DNA probe are completely complementary to the target gene; and a 3′ stem is partially complementary to the 5′ stem; a 5′ terminal and a 3′ terminal are respectively modified by a fluorophore and a quencher group, and partial bases on the loop are modified by locked nucleic acid.

3. The method for simultaneously detecting exosome membrane protein and mRNA of claim 1, wherein the specific DNA probe is self-designed and modified for synthesis according to a target gene sequence; and the fluorescent antibody for labeling the exosome membrane protein is a fluorescein-labeled monoclonal antibody.

4. The method for simultaneously detecting exosome membrane protein and mRNA of claim 1, wherein the specific DNA probe is coated with a cationic lipid composite nanoparticle.

5. (canceled)

6. The method for simultaneously detecting exosome membrane protein and mRNA of claim 1, wherein the sample comprises: cell culture supernatants, isolated laboratory animal plasma, serum, isolated human plasma, serum, urine, and other body fluids or excrement samples.

7. The method for simultaneously detecting exosome membrane protein and mRNA of claim 1, wherein the sample is from a source of a living cell, an animal, human body fluid or an excrement sample.

8-10. (canceled)

11. The method for simultaneously detecting exosome membrane protein and mRNA of claim 2, wherein the specific DNA probe is coated with a cationic lipid composite nanoparticle.

12. The method for simultaneously detecting exosome membrane protein and mRNA of claim 3, wherein the specific DNA probe is coated with a cationic lipid composite nanoparticle.


SEQUENCE LISTING (13 sequences)

SEQ ID NO: 1 - DNA, 36 bp, Artificial Sequence
  Synthesized Probe
cgcgatcggaggatgtgccagaggtagttgatcgcg

SEQ ID NO: 2 - DNA, 35 bp, Artificial Sequence
  Synthesized Probe
cgcgatcgctatggtggtgccgactacagatcgcg

SEQ ID NO: 3 - DNA, 34 bp, Artificial Sequence
  Synthesized Probe
cgcgatctggtgccgactacaagcgaagatcgcg

SEQ ID NO: 4 - DNA, 35 bp, Artificial Sequence
  Synthesized Probe
cgcgatctggtgccgactacaagcgaatgatcgcg

SEQ ID NO: 5 - DNA, 35 bp, Artificial Sequence
  Artificial Sequence
cgcgatcggaggatgtgccagaggtagtgatcgcg

SEQ ID NO: 6 - DNA, 36 bp, Artificial Sequence
  Synthesized Probe
cgcgatcgctatggtggtgccgactacaagatcgcg

SEQ ID NO: 7 - DNA, 35 bp, Artificial Sequence
  Synthesized Probe
cgcgatcggcggcgacttcatgctgcgagatcgcg

SEQ ID NO: 8 - DNA, 38 bp, Artificial Sequence
  Synthesized Probe
cgcgatcgacttcatgctgcgacgcccgtttgatcgcg

SEQ ID NO: 9 - DNA, 35 bp, Artificial Sequence
  Synthesized Probe
cgcgatccgccctgcggaccctgccttcgatcgcg

SEQ ID NO: 10 - DNA, 40 bp, Artificial Sequence
  Synthesized Probe
cgcgatcccagggctgcttgcttgtctgtctcagatcgcg

SEQ ID NO: 11 - DNA, 38 bp, Artificial Sequence
  Synthesized Probe
cgcgatctgccagggctgcttgcttgtctgtgatcgcg

SEQ ID NO: 12 - DNA, 35 bp, Artificial Sequence
  Synthesized Probe
cgcgatcgcgacgcccgtttcccaagccgatcgcg

SEQ ID NO: 13 - DNA, 36 bp, Artificial Sequence
  Synthesized Probe
cgcgatcgctgcgacgcccgtttcccaaggatcgcg